(19) United States
(12) Patent Application Publication
(10) Pub. No.: US 2025/0059485 A1
(43) Pub. Date: Feb. 20, 2025

(54) CO2 FED ALGAE GROWTH AND HARVEST

(71) Applicant: PHYCO2 LLC, Henderson, NV (US)

(72) Inventors: William B. Clary, Newburyport, MA (US); Robert E. Morgan, Henderson, NV (US); Gregory A. Hagopian, Henderson, NV (US); Eric Hagopian, Henderson, NV (US)

(21) Appl. No.: 18/235,718

(22) Filed: Aug. 18, 2023

(51) Int. Cl. C12M 1/00; C12M 1/34

(52) U.S. Cl. CPC C12M 29/06; C12M 31/10; C12M 41/06; C12M 41/40; C12M 47/02

(57) ABSTRACT

Systems and methods are provided for processing carbon dioxide-containing gas based on biomass. Such processing may include collection from a point source or atmosphere, drying, treatment, and compression. The compressed gas may be fed into an algae reactor that maintains optimal conditions for algae growth. The algae may be allowed to grow until a predetermined density associated with harvesting is reached. The harvested algae may be processed and packaged. The process may be monitored by sensors and controlled by a computing device in communication with the devices associated with each stage of processing.

### BACKGROUND OF THE INVENTION

### 1. Field of the Disclosure

The present disclosure is generally related to systems for capturing carbon dioxide-containing gases. More particularly, the present disclosure is related to systems that use biomass to store and utilize the captured carbon dioxide.

### 2. Description of the Related Art

A problem related to carbon capture is the high cost involved in capturing, sequestering, and utilizing the captured carbon dioxide. Building the necessary infrastructure for capturing, transporting, and storing carbon dioxide can be costly from constructing carbon capture facilities to developing pipelines or transportation systems. Furthermore, the operational costs in time, energy, and resources associated with continuous monitoring, maintenance, and energy consumption for carbon capture processes pose a significant challenge and barrier to entry, particularly for industries or regions with limited resources and capabilities, thereby hindering the widespread adoption of carbon capture technologies.

There are also energy intensity and efficiency trade-offs associated with carbon capture as many carbon capture methods, such as post-combustion capture, often require additional energy to operate effectively. For instance, solvent-based capture techniques necessitate heating the solvent to release the captured carbon dioxide. This energy consumption can negatively impact the overall efficiency of power plants or industrial processes, reducing their net energy output.

A further problem is the limited infrastructure for the utilization and storage of captured carbon dioxide. After the capture process, the carbon dioxide needs to be transported and securely stored to prevent its release into the atmosphere. Establishing an extensive network of pipelines, storage facilities, and suitable geological formations for long-term storage requires substantial investments and careful planning. Insufficient infrastructure can result in higher costs and logistical complexities in transporting captured carbon dioxide to storage sites. Additionally, identifying suitable storage sites can be challenging due to geological considerations and potential public resistance to underground storage.

There is, therefore, a need in the art for improved systems and methods for effectively and efficiently capturing carbon dioxide-containing gases.

### SUMMARY OF THE CLAIMED INVENTION

Embodiments of the claimed invention include systems and methods for processing carbon dioxide-containing gas. Such processing may include collection from a point source or atmosphere, drying, treatment, and compression. The compressed gas may be fed into an algae reactor which maintains optimal conditions for algae growth. The algae may be allowed to grow and reach a predetermined level of density associated with initiation of harvesting. The harvested algae may be processed and packaged for sale. The process may be monitored and controlled by various sensors operating in conjunction with computing devices coupled to the devices at each processing stage.

### BRIEF DESCRIPTIONS OF THE DRAWINGS

FIG. 1 illustrates an exemplary network environment in which a system for CO₂ capture and biomass-based storage may be implemented.

FIG. 2 is a flowchart illustrating an exemplary method for CO₂ capture and biomass-based storage.

FIG. 3 is a flowchart illustrating an exemplary method for gas collection.

FIG. 4 is a flowchart illustrating an exemplary method for reactor preparation.

FIG. 5 is a flowchart illustrating an exemplary method for controlling algae growth.

FIG. 6 is a flowchart illustrating an exemplary method for harvesting and processing algae products.

FIG. 7 is a block diagram of an exemplary computing device that may be used to implement an embodiment of the present invention.

### DETAILED DESCRIPTION

Embodiments of the claimed invention include systems and methods for processing carbon dioxide-containing gas. Such processing may include collection from a point source or atmosphere, drying, treatment, and compression. The compressed gas may be fed into an algae reactor which maintains optimal conditions for algae growth. The algae may be allowed to grow and reach a predetermined level of density associated with initiation of harvesting. The harvested algae may be processed and packaged for sale. The process may be monitored and controlled by various sensors operating in conjunction with computing devices coupled to the devices at each processing stage.

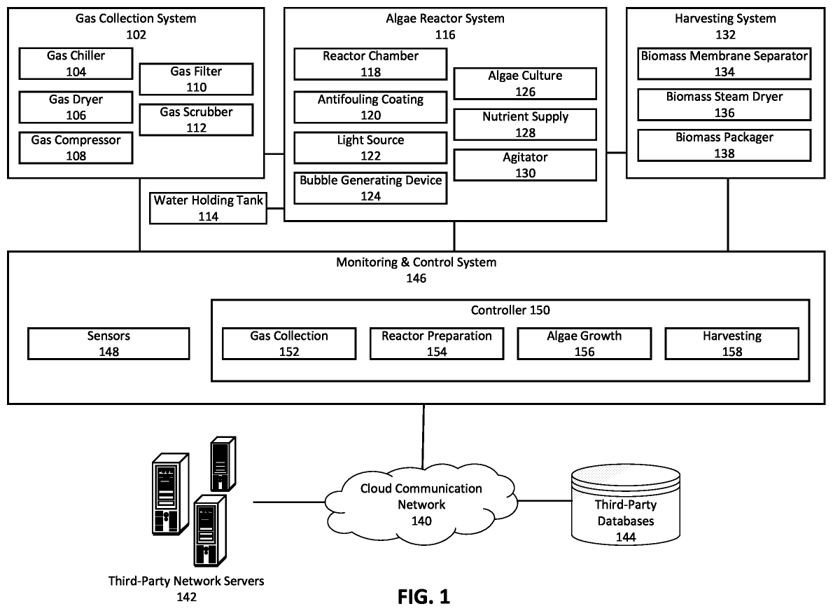

FIG. 1 illustrates an exemplary network environment in which a system for CO₂ capture and biomass-based storage may be implemented. As illustrated, the network environment may include a gas collection system 102, water holding tank 114, algae reactor 116, harvesting system 132, cloud communication network 140, and monitoring and control system 146.

Gas collection system 102 may be configured to operate in conjunction with any source of gas, particularly gases containing carbon dioxide (CO2), including atmospheric or point sources. Point sources of CO2 may include industrial processes such as combustion processes, which involve the burning of fossil fuels for energy generation, heating, or other purposes. Combustion processes in power plants, refineries, and factories release CO2 as a product of the chemical reaction between fuel and oxygen. Concrete production is another significant point source of CO2 emissions. Cement, a key component of concrete, is produced through a process called calcination during which limestone (calcium carbonate) is heated to high temperatures, resulting in the release of CO2 as a byproduct. Metallurgical processes such as iron and steel production release CO2 as a byproduct of the reduction of iron ore using carbon (coke). Gas collection system 102 may be arranged and connected to one or more gas sources described herein using various conduits, channels, compressors, etc., for directing and controlling flow of gas. As illustrated, gas collection system 102 may include gas chiller 104, gas dryer 106, gas compressor 108, gas filter 110, and gas scrubber 112.

Gas chiller 104 first directs gas to a heat exchanger, which facilitates the transfer of heat from the gas to a cooling medium. The cooled gas may then enter a gas compressor 108, which increases its pressure for subsequent cooling and liquefaction. The gas may then be directed through a series of condensers, expansion valves, and evaporators. As the gas flows through the gas chiller 104 and exchanges heat with a refrigerant which circulates through a closed loop. The refrigerant absorbs the heat from the gas, causing it to cool down. The specific design and configuration of the gas chiller 104 depends on factors such as the volume and temperature of the gas, as well as the specific requirements of the application. Different cooling techniques, such as indirect cooling using refrigerants, are employed to achieve the desired cooling and liquefaction of the gas.

Gas dryer 106 removes water vapor from a gas, particularly a gas containing CO2. In an embodiment, a gas dryer 106 may be a water dropout filter or a desiccant dryer. This type of dryer typically includes a vessel filled with a desiccant material, such as silica gel or activated alumina. The gas containing CO2 is directed into the dryer vessel, where it comes into contact with the desiccant. The desiccant material has a high affinity for water molecules, so as the gas passes through the dryer, the desiccant adsorbs the moisture from the gas. This adsorption process effectively removes the water vapor, leading to a reduction in the humidity of the gas. The dried gas then exits the gas dryer 106. To maintain the efficiency of the drying process, desiccant dryers often include regeneration heaters or blowers, which periodically regenerate the desiccant material by heating the desiccant to remove the absorbed moisture, allowing it to be reused for subsequent drying cycles. The specific design and configuration of the gas dryer 106 may vary based on factors such as the flow rate and temperature of the gas, as well as the desired moisture content. Additionally, for larger-scale industrial applications, specialized drying systems can be employed, such as adsorption dryers or membrane dryers, which offer more advanced moisture removal capabilities.

Gas compressor 108 may include various stages or cylinders. Each cylinder contains a piston or impeller that oscillates, reducing the volume of the gas and increasing its pressure. As the piston or impeller moves, it compresses the gas within the cylinder. This compression process involves reducing the space available for the gas to occupy, causing its molecules to come closer together increasing the density and pressure of the gas. The gas is progressively compressed as it passes through multiple stages or cylinders, with each stage contributing to a further increase in pressure. Gas compressors 108 can be driven by various sources of power, such as electric motors or engines. Depending on the application, different types of compressors may be utilized, including reciprocating compressors, rotary screw compressors, or centrifugal compressors. The specific choice of compressor depends on factors such as the desired compression ratio, flow rate, and efficiency requirements. The compressed gas containing CO2 can be utilized for various purposes, including storage, transportation, or further processing. Higher pressure enables more efficient storage or transport of the gas, reducing the required storage or transportation volume.

Gas filter 110 removes particulates and other contaminants from a gas stream, such as a stream of gas containing CO2. Gas filter 110 may include one or more filters designed to capture undesired particles and contaminants present in the gas. The filters used may vary depending on the nature and size of the particles to be removed. Common filter types include particulate filters, bag filters, cartridge filters, electrostatic precipitators, etc. The gas filter 110 is designed with porous media that allows the gas to pass through while trapping the particulates and contaminants. These trapped particles can include dust, ash, soot, or other solid impurities present in the gas stream. The filtration process operates based on various mechanisms, such as mechanical sieving, interception, or diffusion. Larger particles are typically captured by mechanical sieving, as they are unable to pass through the small openings in the filter media. Smaller particles may be removed through interception, where they collide with and adhere to the filter fibers. Diffusion captures ultrafine particles by causing them to collide and stick to the filter media due to Brownian motion. Regular maintenance and periodic replacement of filters may be necessary to ensure optimal filtration efficiency. Over time, the filters may become clogged with captured particles, reducing their effectiveness. By replacing or cleaning the filters, the filtration system can continue to efficiently remove particulates and contaminants from the gas stream. Removing particulates and contaminants helps protect downstream equipment and processes from damage or fouling.

A gas scrubber 112 typically includes a vessel or tower filled with a scrubbing solution, such as water or a chemical reagent. The gas to be treated is directed into the scrubber, where it comes into contact with the scrubbing solution. As the gas flows through the gas scrubber 112, chemical reactions occur between the contaminants in the gas and the scrubbing solution. These reactions result in the absorption or neutralization of the contaminant chemicals or components present in the gas. The scrubbing solution acts as a medium to capture or react with the contaminants, effectively removing them from the gas. The specific design of the gas scrubber 112 can vary based on the contaminants and chemicals present in the gas. Different types of gas scrubbers 112, such as wet scrubbers or dry scrubbers, may be utilized depending on the requirements of the gas treatment process. In addition to the scrubbing solution, the gas scrubber 112 may also incorporate additional components such as mist eliminators or demisters. These components help remove any liquid droplets or mist formed during the scrubbing process, ensuring that the treated gas leaving the gas scrubber 112 is as free from contaminants as possible. Gas scrubber 112 helps create a suitable and safe gas stream for algae cultivation.

Water holding tank 114 is a storage vessel designed to hold water received from various sources such as wells, municipal sources, water treatment facilities, or as a byproduct of industrial processes like drying a CO2-containing gas or separating water from an algae slurry. Water holding tank 114 is typically constructed using materials that are resistant to corrosion and contamination, such as concrete, steel, or plastic. The primary function of a water holding tank 114 is to store water for later use or to facilitate the management and distribution of water within a system such as an algae reactor 116. The water holding tank 114 is equipped with inlet and outlet connections, allowing water to enter and exit as needed. The inlet connection is connected to one or more water sources, while the outlet connection provides a means for drawing water from the tank. Water holding tank 114 may include various components such as level sensors 148 or float switches to monitor and control the water level, pressure regulators to maintain consistent water pressure, and valves for controlling the flow of water in and out of the water holding tank 114.

Water holding tank 114 operates based on the principle of maintaining a balance between water inflow and outflow. When water enters the water holding tank 114 from a source, it fills the water holding tank 114 until it reaches a predetermined level. Once the water holding tank 114 is full, the inflow is regulated, and the excess water may be redirected back to the source, to other parts of the system, or may be expelled to a drain or to the environment. In some embodiments, the water may be treated via UV light and/or one or more filters to kill and/or remove bacteria and other live organisms in the water. The water may be treated to remove metals and transition elements, such as by an ion exchange filter.

An algae reactor 116, also known as a photobioreactor, is a specialized system designed to cultivate algae using a CO2-containing gas, such as a CO2-containing waste gas from an industrial process or combustion. As illustrated, algae reactor 116 may include a reactor chamber 118, antifouling coating 120, light source 122, bubble generating device 124, algae culture 126, nutrient supply 128, and agitator 130.

The algae reactor may include a reactor chamber 118 that allows light to penetrate a growth medium and supports the growth of algae. Within the reactor chamber 118, an algae slurry or suspension is maintained, which serves as the growth medium for the algae. The CO2-containing waste gas may be fed through a bubble generating device 124 which evenly disperses the CO2-containing gas into the algae slurry and may further use bubble lift processes to achieve fluid circulation and mixing while ensuring efficient contact between the CO2 and the algae cells. This CO2-containing gas serves as the carbon source for algae photosynthesis.

Reactor chamber 118 provides a controlled environment for the cultivation of algae. The reactor chamber 118 may be a sealed container or vessel and may be made of transparent material, such as glass or plastic, which may allow light to penetrate into the chamber. The algae reactor may also include metal materials, such as iron, steel, aluminum, etc. to facilitate heat transfer. The reactor chamber 118 may contain mixing mechanisms or agitators 130, such as stirrers or circulation pumps, to ensure even distribution of the algae and nutrients throughout the reactor chamber 118.

An antifouling coating 120 may prevent the attachment and growth of unwanted organisms, such as biofilms, algae, or other fouling agents, on the surfaces of the algae reactor 116 components. The antifouling coating 120 may include a specialized material or chemical formulation that inhibits the adhesion and colonization of fouling organisms. The antifouling coating 120 may be applied to the surfaces of the reactor chamber 118, including the transparent vessel, gas delivery system, and other relevant components. In some embodiments, the surfaces contacting the algae slurry possess antifouling characteristics and may not require an antifouling coating 120. An antifouling coating 120 may include biocides or antimicrobial agents. These agents may be incorporated into the coating formulation and may release over time to deter the growth of microorganisms and algae. The biocides act by disrupting cellular processes or inhibiting the colonization of fouling organisms, thereby preventing their attachment and growth on the coated surfaces. An antifouling coating 120 may modify surface properties such as surface energy or roughness, to create an unfavorable environment for fouling organisms. For example, hydrophobic coatings can reduce the ability of waterborne organisms to adhere to the surface, making it more difficult for them to establish and grow.

An algae reactor 116 may also be equipped with one or more light sources 122, such as LED panels, strips, tubes, or fluorescent lamps, to provide the necessary illumination for photosynthesis. The intensity and duration of light exposure may be controlled to optimize algae growth and productivity. A light source 122 provides the necessary illumination for photosynthesis, supporting the growth and productivity of the algae. The light source 122 in an algae reactor may emit specific wavelengths of light that are optimal for photosynthetic activity. LED panels or fluorescent lamps are commonly used as light sources due to their energy efficiency and controllable light spectrum. These light sources 122 can be positioned around and/or within the reactor to ensure uniform distribution of light throughout the algae slurry. The light source 122 operates based on the principle of converting electrical energy into light energy. In the case of LEDs, electric current passes through a semiconductor material, causing the release of photons that generate light. Fluorescent lamps work by passing an electric current through a gas-filled tube, which results in the excitation of mercury atoms and the subsequent emission of ultraviolet light. This ultraviolet light is then converted into visible light through a phosphor coating on the inner surface of the lamp. The intensity and duration of light exposure in the algae reactor may be controlled to optimize algae growth and productivity. Light intensity may be adjusted by controlling the power supply or dimming capabilities of the light source and cycling the light for certain periods and durations either with a sign wave or square wave profile. Additionally, timers or programmable controllers may be used to regulate the duration and timing of light exposure, simulating day and night cycles to maintain proper growth conditions for the algae or in an alternate pattern and/or frequency to optimize algae growth.

A bubble generating device 124 facilitates efficient gas-liquid contact between the CO2-containing waste gas and the algae slurry by dispersing bubbles of the gas within the algae culture 126 to enhance the transfer of CO2 to the algae cells. The bubble generating device 124 may additionally act to cool the liquid via the injection of cool bubbles. The bubble generating device 124 typically may include a microbubbler, diffuser, or membrane, which produces fine bubbles. A CO2-containing gas may flow through the microbubbler encountering small pores or perforations in the material, causing the gas to be released in the form of numerous small bubbles. These bubbles rise to the surface of the algae slurry, creating a gas-liquid interface where the CO2 can be efficiently absorbed by the algae cells. In some embodiments, at least some of the CO2 is solubilized into the water of the algae slurry. The rising action of the bubbles may additionally perform the function of agitating the algae. The bubble generating device 124 may include a distribution system to ensure even dispersion of the bubbles throughout the algae slurry.

An algae culture 126 is a population of algae cells which are prepared for growth within an algae reactor 116. The algae culture in algae reactor 116 is typically maintained as an algae slurry or suspension. The slurry contains water, nutrients, and the algae cells. The water serves as the medium for supporting algae growth, providing the necessary environment for their survival and reproduction. The algae culture 126 may be processed to remove microorganisms, such as via use of UV light, and other harmful elements, which may inhibit algae growth or compromise the efficiency of algae growth in an algae reactor 116.

The algae reactor 116 may incorporate a nutrient supply 128 and/or delivery system to deliver solutions containing essential elements like nitrogen, phosphorus, and potassium which may be added to the algae slurry to support algae growth The nutrient concentrations may be monitored and adjusted to maintain an optimal balance for algae cultivation. The algae reactor 116 may include a temperature regulation system to maintain the desired temperature range for optimal algae growth. This may be achieved through heating or cooling mechanisms, such as heat exchangers or temperature controllers. The reactor chamber 118 may incorporate a light source 122 to provide the necessary illumination for photosynthesis. Light-emitting diodes (LEDs) or fluorescent lamps are commonly used to deliver the specific wavelengths and intensities of light required for optimal algae growth. The light source is positioned in a way that evenly distributes light across the algae slurry, ensuring uniform exposure for all algae cells. The light source may be located within the reactor chamber 118.

A nutrient supply 128 provides the necessary elements for the growth and metabolic processes of the algae culture 126. The nutrient supply 128 may include a controlled delivery of essential nutrients to the algae slurry which may include nitrogen (N), phosphorus (P), potassium (K), and other trace elements. The nutrient supply 128 may include nutrient solutions and/or additives that contain predetermined concentrations of nutrients. The nutrient supply 128 may include reservoirs or tanks that hold the nutrient solutions, dosing pumps and/or injectors that deliver the nutrients into the algae slurry, and monitoring devices to ensure control of nutrient concentrations. The dosing pumps and/or injectors may be programmed to deliver the nutrients at specific intervals or according to a predetermined schedule. The nutrient supply 128 system may additionally incorporate sensors 148 to monitor and adjust nutrient levels. An example of a nutrient supply 128 may include a pH supply and/or delivery system to deliver solutions containing essential ions and buffers to maintain a pH level which may be added to the algae slurry to support algae growth. In an embodiment, a nutrient supply may include a chelating supply and/or delivery system to deliver solutions containing essential chemical components and ions to reduce the metals and nutrients from the algae cell walls during harvesting and to support algae growth.

An agitator 130 mixes and circulates the algae slurry, facilitating an even distribution of algae throughout the growth medium. The agitator 130 may include a motor or drive unit, which provides the necessary rotational force for the agitator. The motor may be connected directly, or indirectly via a shaft to one or more impellers or blades. The impellers or blades create turbulence and induce fluid motion within the algae reactor, ensuring uniform distribution of nutrients, gases, and light exposure to the algae culture 126. The shape and arrangement of the impellers may vary. The agitator 130 helps distribute nutrients evenly, ensuring uniform growth and preventing nutrient depletion or buildup in certain areas of the reactor. In some embodiments, the agitator 130 may not include a physical mechanism, but instead the effect may be achieved via the rising action of bubbles of CO2-containing gas through the algae slurry.

A harvesting system 132 separates the algae biomass from the liquid medium and may utilize mechanical centrifuge, membranes, settling zones or baffles to increase the concentration of the algae cells. The algae slurry may be allowed to rest or undergo gentle agitation, which promotes the settling of algae cells to the bottom of the vessel. As the algae cells settle, a concentrated layer of biomass forms, while the liquid phase (supernatant) containing water and other components is decanted or drained off. The concentrated algae biomass may be further dewatered using techniques such as filtration, centrifugation, or flocculation. Filtration involves passing the algae slurry through a filter medium that retains the algae cells while allowing the liquid to pass through. Centrifugation utilizes centrifugal force to separate the denser algae cells from the liquid phase. Flocculation involves the addition of chemical agents to induce the clumping of algae cells, facilitating their precipitation and separation from the liquid. As illustrated, harvesting system 132 may include biomass membrane separator 134, biomass steam dryer 136, and biomass packager 138.

In some implementations, the algae slurry is removed from the algae reactor 116 and most of the water is removed via the use of a biomass membrane separator 134. The water content of the algae may further be reduced via the use of a biomass steam dryer 136. A biomass membrane separator 134 may include a semi-permeable membrane having specific pore sizes that allow for the passage of liquid, dissolved substances, and smaller molecules while retaining the larger algae cells and biomass. The liquid phase, which contains water and smaller dissolved molecules, passes through the membrane and the water may be recovered and stored in a water holding tank 114. Via a combination of size exclusion and molecular sieving the algae cells, which are larger in size, are unable to pass through the small pores or openings of the membrane, while allowing the liquid components can permeate through the membrane structure due to their smaller size. The biomass membrane separator 134 may incorporate additional mechanisms such as backwashing or crossflow filtration. Backwashing involves periodically reversing the flow direction across the membrane surface to dislodge and remove accumulated particles and debris. Crossflow filtration utilizes a tangential flow of liquid across the membrane surface, preventing the buildup of fouling materials and maintaining optimal filtration performance.

A biomass steam dryer 136 may include a drying chamber or vessel, a steam generator, and a heat exchanger. The harvested algae biomass is introduced into the drying chamber, which is designed to facilitate the drying process. The steam generator generates high-pressure steam, which raises the temperature within the drying chamber, facilitating the evaporation of moisture from the algae biomass. As the algae biomass is exposed to the heated environment, the moisture within the cells begins to vaporize and then condenses on the cooler surfaces within the drying chamber, such as condensation plates or heat exchange coils before being collected and removed from the system. The drying process continues until the desired moisture content is achieved, resulting in a dry and concentrated algae biomass product. The dried biomass can then be further processed or utilized for various applications.

A biomass packager 138 may include a packaging machine or system, which may be automated or semi-automated and may include a conveyor belt, feeding mechanism, weighing system, and sealing apparatus The harvested algae biomass is fed into the biomass packager 138 by a conveyor belt or feeding mechanism. The algae is weighed to determine the desired packaging weight or quantity and is then deposited into the packaging material, such as bags or containers. The biomass packager 138 seals the package using any of heat sealing, adhesive sealing, or other mechanisms to ensure proper closure and preservation of the algae biomass. The packaging may include any of bulk packaging, individual bags, or containers of varying sizes.

A cloud communication network 140 may include a network of remote servers that are interconnected and provide various services and resources over the Internet. Cloud communication network 140 may include network infrastructure, servers, storage systems, and software applications. The network infrastructure allows for seamless connectivity and communication between the algae reactor and the cloud communication network 140 servers. The servers host the necessary software applications and algorithms required for data processing, analysis, and control. The storage systems provide secure and scalable storage capacity for data generated by the algae reactor. Cloud communication network 140 may facilitate remote control and management of an algae reactor. Operators may access a cloud-based control panel or dashboard to monitor and adjust various parameters of the reactor, such as CO2 flow rate, nutrient, pH, chelating agent concentration, algae density, temperature, electricity consumption, and lighting conditions. Cloud communication network 140 also enables the storage and retrieval of historical data, analysis, and future optimization of the algae reactor 116 performance. As illustrated, cloud communication network 140 may further allow for communication with third-party network servers 142 and third-party databases 144.

A third-party network server 142 may be inclusive of an external network or platform that is utilized to enable functionality and capabilities of the algae reactor 116. A third-party network servers 142 may offer various services, data analysis tools, and/or additional data or resources to optimize the algae cultivation process and improve CO2 utilization. A third-party network servers 142 may include a cloud-based platform, data analytics tools, and external APIs (Application Programming Interfaces). Data analytics tools within the third-party network servers 142 may enable advanced data processing and insights generation. These tools may employ algorithms and machine learning techniques to analyze the data collected from the algae reactor to provide valuable insights into such as CO2 consumption rates, optimal nutrient dosage, growth patterns, and other performance indicators. These insights can be used to optimize the operation of the algae reactor 116, improve CO2 utilization efficiency, and improve overall productivity. Furthermore, the third-party network servers 142 may offer external APIs that allow seamless integration between the algae reactor and external systems or services. These APIs enable data exchange and interoperability, facilitating collaborations with environmental monitoring agencies and/or industry partners including validation of carbon collected and stored within the produced algae biomass.

A third-party database 144 is any external database which may store data including operational parameters, environmental conditions, growth metrics, and historical records relating to an algae reactor. The third-party database 144 may store data related to growth characteristics of an algae culture 126 created by a vendor who prepares algae cultures 126. Similarly, a third-party database 144 may include manufacturer information relating to any component of an algae reactor 116 including gas collection and biomass harvesting.

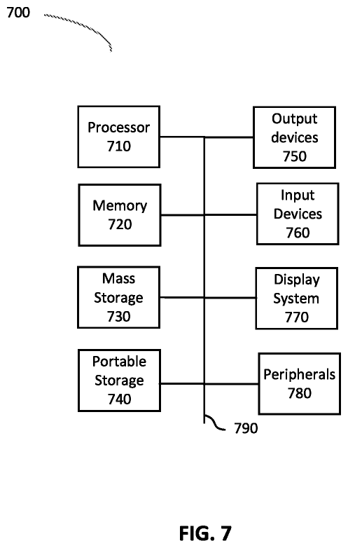

A monitoring and control system 146 for an algae reactor 116 may be inclusive of a computing device, such as described in detail in relation to FIG. 7. Monitoring and control system 146 may include or be in communication with sensors 148 within the algae reactor 116. The sensors 148 measure parameters such as CO2 concentration, temperature, pH level, dissolved oxygen, nutrient levels, etc. The sensors 148 may continuously collect data, providing real-time information about the status of the algae reactor 116. The collected data from the sensors 148 may be transmitted to a central control unit or system. The control unit processes and analyzes the data and may modify one or more parameters of an algae reactor 116 in response to the data collected from one or more sensors 148. For example, the monitoring and control system 146 may control the flow rate of the CO2-containing waste gas, adjust the lighting intensity and duration, regulate the nutrient dosing, monitor the overall conditions of algae reactor 116, etc. The control unit may further communicate with a user interface which displays sensor 148 readings and other relevant information. Sensors 148 collect data by measuring one or more physical properties such as CO2 concentration, temperature, pH level, dissolved oxygen, algae density, and nutrient levels, etc.

CO2 sensors 148 are essential for measuring the concentration of CO2 in the algae reactor 116 and may utilize infrared or electrochemical technology to detect and quantify the CO2 concentration in the gas phase by measuring the absorption or electrical properties of a gas sample and converting it into a CO2 concentration value. Temperature sensors 148 may include thermocouples, resistance temperature detectors (RTDs), optical temperature sensors, etc. pH sensors 148 monitor the acidity or alkalinity of an algae slurry and may utilize pH-sensitive electrodes and/or indicators that generate electrical signals corresponding to the hydrogen ion concentration in the liquid medium. Nutrient sensors 148 may monitor the levels of essential nutrients such as nitrogen, phosphorus, and potassium within the algae reactor 116.

Controller 150 may include a variety of control processes, rules, algorithms, software, services, APIs, etc., related to gas collection 152, reactor preparation 154, algae growth 156, and harvesting 158. Controller 150 and its constituent control processes 152-158 may be executed by a processor (such as described in further detail in relation to FIG. 7) to perform the functions described herein. Controller 150 may make calls to and initiate specific functions associated with the control processes 152-158 as needed, e.g., in response to specific calls, triggers, thresholds, determinations, etc. The controller 150 initiates the gas collection control processes 152, which are executed to collect a CO2-containing gas and process the gas such as by chilling, drying, and compressing the gas prior to transport and/or storage. The CO2-containing gas may additionally be filtered and/or treated to remove particulates, chemicals, and other contaminants. The compressed CO2-containing gas is received, and the reactor preparation control processes 154 are initiated and executed. The reactor preparation control processes 154 receive operational data related to the operation of an algae reactor 116 and prepares reactor surfaces, such as via sterilization and/or application of an antifouling coating. The monitoring and control system 146 is initialized and the algae reactor 116 is filled with a medium, such as water, and an algae culture 126. A reactor status is received and the algae growth control processes 156 are executed to provide a compressed CO2 gas to the algae reactor 116.

One or more sensors 148 are polled for reactor status and measurements, which may be used to determined if adjustments to the levels of any of CO2, nutrients, water, etc., may be necessary. Further, it may be determined whether the algae is ready for harvest, and an algae growth status is received. If the algae is not ready for harvest and the reactor status is nominal, then initiate the algae growth control processes 156. If the algae is ready for harvest, the harvesting control processes 158 is initiated and executed to obtain harvest data including a processing procedure and/or parameters for a processed algae. The algae slurry is collected, and water is separated such as by using a biomass membrane separator 134 and/or a biomass steam dryer 136. The dried algae is measured, packaged, and prepared for shipment. A harvest status is received and when complete, the CO2 capture, algae growth, and harvest system is ended.

The gas collection control processes 152 may be initiated based on a call by the controller 150. Gas collection control processes 152 may be executed to chill a collected CO2-containing gas using a gas chiller 104 and to remove moisture from the air via a gas dryer 106 prior to compressing the gas. The gas is then transported and/or stored for use in an algae reactor 116. The CO2-containing gas may additionally be filtered and/or treated to remove particulates, chemicals, and other contaminants. The compressed CO2-containing gas is sent to the controller 150.

The reactor preparation control processes 154 may be executed to obtain operational data related to the operation of an algae reactor 116 from the controller 150. The operational data indicating one or more parameters related to the growth of algae which may include an algae culture 126, light intensity, nutrient levels to maintain, etc. The algae reactor 116 surfaces are prepared which may include sterilization and/or application of an antifouling coating. Further a monitoring and control system 146 may be initialized, which may further include the initialization of one or more sensors 148 for measuring physical parameters of the algae reactor 116 such as temperature, algae concentration, concentrations of CO2 and/or one or more nutrients, etc. An algae culture 126 may additionally be prepared, which may include acquisition from a third-party and/or UV sterilization to remove contaminants such as microbes which may inhibit algae growth in the algae reactor 116. The reactor is then filled with a medium, such as water, and the algae culture 126. A reactor status is then sent to the controller 150 indicating that the algae reactor 116 is ready to begin growing algae. Alternatively, the reactor status may indicate remaining actions necessary to prepare the algae reactor 116 for algae growth.

The algae growth control processes 156 may be executed to obtain a status of compressed CO2-containing gas from the controller 150. The gas may be distributed into an algae reactor 116 to facilitate algae growth. One or more sensors 148 may be polled to measure physical parameters related to algae growth. If CO2 is needed, the rate of compressed CO2-containing gas released into the reactor chamber 118 via a bubble generating device 124 may be increased. In some embodiments, the compressed CO2-containing gas may be constantly released into the reactor chamber 118. In other embodiments, the compressed CO2-containing gas may alternate between an active release state and an inactive release state. Further, it may be determined whether additional nutrients are needed, such as nitrogen, phosphorus, potassium, and/or one or more of any trace nutrients. Similarly, pH may indicate the need for additional nutrients and/or chemicals to be added to alter physical parameters of the algae slurry. If additional nutrients are needed, they are added to the algae reactor. Similarly, if water is needed, additional water may be added, such as from a water holding tank 114. The added water may be recovered from other processes related to the drying of the CO2-containing gas and/or drying of harvested algae. If the algae is ready for harvest, the reactor status is ready for harvest, otherwise the reactor status is nominal. In some embodiments, the reactor status may indicate an error condition, such as a physical parameter is out of an acceptable range, a contaminant has been detected, a part of the algae reactor has failed, etc. The reactor status is sent to the controller 150.

The harvest control processes 158 may be executed to obtain harvest data from the controller 150. The harvest control processes 158 may include one or more processes and/or parameters of a processed algae. The algae slurry is collected from one or more algae reactors 116 and the medium, such as water, is separated from the algae slurry. In an embodiment, the water may be separated from the algae using a biomass membrane separator 134. The algae may be further dried such as by using a biomass steam dryer 136. Water recovered via a biomass membrane separator 134 and/or biomass steam dryer 136 may be stored in a water holding tank 114 for use elsewhere during the process. The dried algae is measured and packaged, prepared for shipment, and a harvest status is sent to the controller 150.

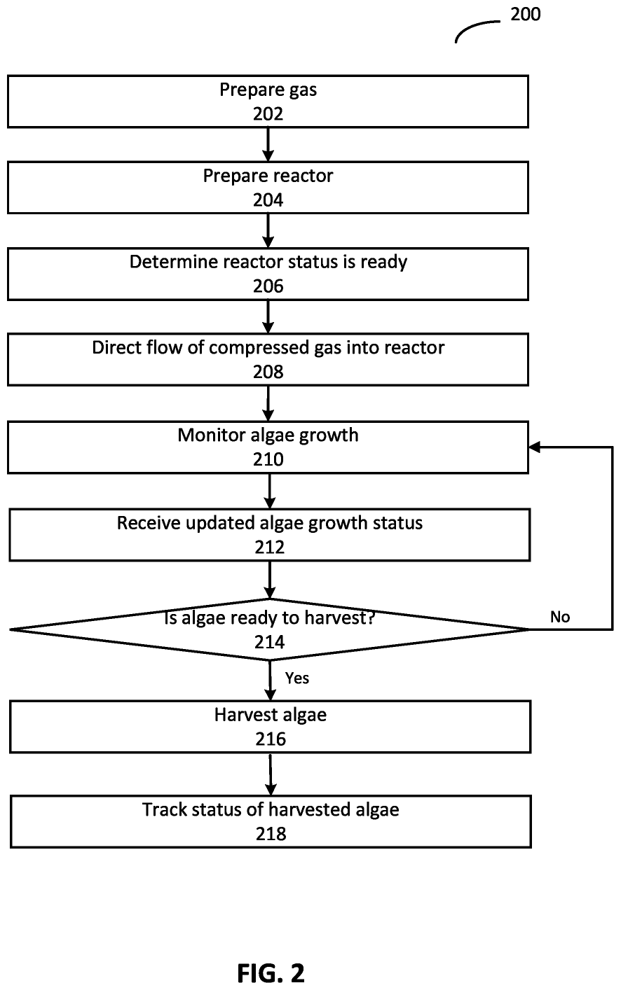

FIG. 2 is a flowchart illustrating an exemplary method for CO2 capture and biomass-based storage. The method of FIG. 2 may be performed based on execution of controller 150 by a processor of a computing device. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

At step 202, gas may be prepared by the gas collection control processes 152, which may be initiated and called by controller 150 to collect a CO2-containing gas from any of the atmosphere, an industrial process, fermentation, and/or combustion. The CO2-containing gas is chilled, moisture is removed from the CO2-containing gas, and the CO2-containing gas may optionally be filtered and/or treated before being compressed.

At step 204, the reactor may be prepared by reactor preparation control processes 154, which may be initiated by controller 150. The reactor preparation control processes 154 may be executed to obtain operational data relating to the operational parameters of an algae reactor 116 which may include the growth characteristics of an algae culture 126. The reactor surfaces are prepared, such as by applying an antifouling coating 120 and the reactor control system, which may include a monitoring and control system 146 including one or more sensors 148, is initialized. An algae culture 126 is prepared, and the algae reactor 116 is filled with a growth medium, such as water, the algae culture 126, and may further be supplied one or more of nutrients from a nutrient supply 128, and/or a CO2-containing gas.

At step 206, a reactor status may be received by controller 150 in accordance with the reactor preparation control processes 154. The reactor status indicates whether the algae reactor 116 is ready to initiate growth, including the monitoring and control of the algae growth process. Where the reactor status may alternatively indicate that the reactor is not ready to initiate growth, the reactor status may indicate an error condition that must first be resolved.

At step 208, a flow of compressed gas may be directed into the algae reactor 116 in accordance with the gas collection control processes 152. The compressed gas having additionally been cooled, had its moisture content reduced, and optionally being filtered and/or treated to remove one or more contaminants. The compressed gas may be stored and/or transported to an algae reactor 116.

At step 210, the algae growth control processes 156 may be initiated based on a call by controller 150 to monitor the status of algae growth. The algae growth control processes 156 may be executed to obtain a status of a CO2-containing compressed gas, which may include polling one or more sensors 148. The one or more sensors indicating whether CO2 is needed, and if needed, adding more CO2-containing gas to the algae reactor 116. The sensors 148 additionally measuring at least one nutrient concentration, and if more nutrient is needed, adding more nutrient to the algae reactor 116. The one or more sensors 148 further measuring the volume of the slurry in the algae reactor 116 and determining whether more water is needed, and if so, adding more water. The one or more sensors 148 also measuring whether the algae is ready for harvest, and if so indicating that the reactor status is ready for harvest, otherwise the reactor status is nominal.

At step 212, an updated reactor status indicating algae growth status may be received by controller 150 in accordance with the algae growth control processes 156. The reactor status may indicate whether the algae in the algae reactor 116 is ready for harvest or the reactor status is nominal, undergoing growth. The reactor status may further include an error condition such as detection of an undesirable microorganism, malfunction of one or more sensors, depletion of nutrients, etc. In an embodiment, the algae growth status may be ready for harvest. In an alternate embodiment, the algae growth status may be nominal.

At step 214, a determination may be made as to whether the algae is ready for harvest. The algae is ready for harvest when the algae growth status indicates that the algae is ready for harvest, typically when the density of the algae reaches a threshold value or alternatively when the growth rate of the algae declines. If the algae is determined not to be ready, the method may return to step 210 in which the algae growth continues to be monitored. If the algae is determined to be ready, however, the method may proceed to step 216.

At step 216, the harvesting control processes 158 may be initiated by controller 150. The harvest control processes 158 may be executed to obtain harvest data including a processing procedure and/or parameters, such as a target moisture content. The algae slurry is collected, and a majority of the water is separated from the algae before further drying the algae. The algae is then measured and packaged, which may include analysis of properties of the algae. The algae is then prepared for shipment which may include placing the packaged algae on a pallet and/or a vehicle for transport. Alternatively, the algae may be stored for a period of time prior to shipment.

At step 218, a harvest status may be received and tracked by controller 150 in accordance with the harvest control processes 158. The harvest status may indicate a quantity of algae which has been harvested, processed, and prepared for shipment. The harvest status may further include data from analysis of the processed algae which may at least include a moisture content of the algae and may further include properties such as protein, carbohydrate, and/or lipid content.

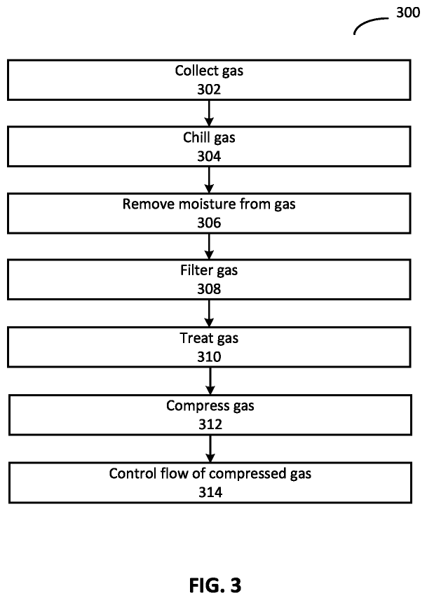

FIG. 3 is a flowchart illustrating an exemplary method for gas collection. The method of FIG. 3 may be performed based on execution of gas collection control processes 152 by a processor in accordance with initiation and call by controller 150.

The process begins at step 302, in which gas collection is initiated for a CO2-containing gas from a gas collection system 102 including any of atmosphere, an industrial process, and/or combustion. Industrial processes may include exhaust gases from combustion, such as from a power plant which may utilize combustion of hydrocarbon fuels such as coal, natural gas or methane, propane, wood or other biomass, etc. Industrial processes may further include metallurgy, concrete production, fermentation, etc. where CO2 is produced. In an embodiment, the CO2-containing gas may be an exhaust from a coal power plant. In an alternate embodiment, the CO2-containing gas may be the result of a chemical reaction in which calcium carbonate is calcinated and converted to lime, the primary component of cement. The CO2-containing gas may be collected by gas collection system 102 from more than one gas source systems. In some embodiments, the CO2 may be captured using an adsorption/desorption process.

At step 304, a CO2-containing gas may be chilled via a gas chiller 104. A gas chiller may utilize a heat exchanger to remove heat from the gas. A heat exchanger may include a refrigerant, which may be a synthetic compound or water, which is a cooler temperature than the collected CO2-containing gas. The heat is transferred to the cooler refrigerant which may be isolated from the CO2-containing gas which increases the temperature of the refrigerant. The refrigerant may utilize radiators, compressors, etc. to cool the refrigerant before recycling the refrigerant.

At step 306, the moisture may be removed from the CO2-containing gas. Moisture can be removed from a CO2-containing gas by cooling the CO2-containing gas so that the water condenses out of the CO2-containing gas. Similarly, the pressure may be increased until the water condenses out of the CO2-containing gas. Alternatively, desiccants may be used to absorb the water molecules from the CO2-containing gas. In an embodiment, the CO2-containing gas may be cooled, causing moisture within the CO2-containing gas to condense, allowing it to be drained away from the CO2-containing gas. In another embodiment, the CO2-containing gas may contact a desiccant, which absorbs the water from the CO2-containing gas. If a desiccant is used, it may be heated to regenerate the desiccant, allowing it to be reused.

At step 308, a CO2-containing gas may be filtered to remove particulates and other contaminants. Filtering a CO2-containing gas may be optional. In some embodiments, the CO2-containing gas is the result of a process where the composition of the CO2-containing gas is known and further is known not to contain particulates and/or contaminants which must be removed via filtration. In alternate embodiments, a gas filter 110, which may include activated carbon, is used to remove particulates, such as ash resulting from the combustion of coal. In an alternate embodiment, a gas filter 110 may not be necessary, as the CO2-containing gas is the result of the combustion of methane or other combination of low carbon number hydrocarbon gases. In some embodiments, filtering a gas may occur immediately after collection, before the gas is cooled, moisture removed, etc.

At step 310, a CO2-containing gas may be treated to remove molecules, chemicals, and other contaminants. Treating a CO2-containing gas may be optional. In some embodiments, the CO2-containing gas is the result of a process where the composition of the CO2-containing gas is known and further is known not to contain any molecules, chemicals, or other contaminants which may be harmful to an algae culture 126. In alternate embodiments, a gas scrubber 112, which may include one or more chemicals or compounds for reacting with undesirable molecules, chemicals, and other contaminants, is used to remove the undesirable components, such as resulting from the combustion of coal. For example, combustion of coal and biomass may result in the production of nitrates, sulfates, and other metal oxides which may create an acidic or caustic environment if allowed to solubilize in water. In an alternate embodiment, a gas scrubber 112 may not be necessary, as the CO2-containing gas is the result of the combustion of methane. In some embodiments, treating a gas may occur immediately after collection, before the gas is cooled, moisture removed, etc.

At step 312, a CO2-containing gas may be compressed. Compressing the CO2-containing gas allows larger amounts of the CO2-containing gas to be stored and/or transported in the same volume of space. The increased pressure may additionally be required to facilitate transit of the CO2-containing gas through a bubble generating device 124. In an embodiment, the CO2-containing gas may be compressed to a pressure of 10 psi.

At step 314, a flow of the compressed CO2-containing gas may be directed by the controller 150 (e.g., into the algae reactor 116). In an embodiment, the CO2-containing gas may be collected from the combustion of methane, is cooled, moisture content reduced to no more than 5% by weight and is compressed to 10 psi. In an alternate embodiment, the CO2-containing gas may be collected from the combustion of coal, is cooled, moisture content reduced to no more than 10% by weight and is further filtered and treated via a gas scrubber 112, prior to being compressed to 50 psi. A flow of the compressed gas may thereafter be directed at a specified rate of flow by controller 150 and gas collection control processes 152.

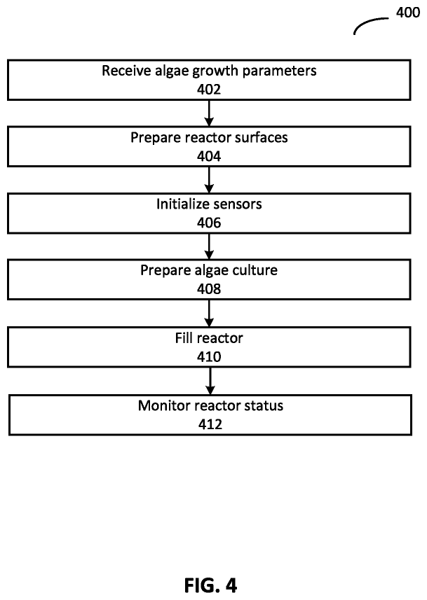

FIG. 4 is a flowchart illustrating an exemplary method for reactor preparation. The method of FIG. 4 may be performed based on execution of reactor preparation control processes 154 by a processor accordance with initiation and call by controller 150.

The process begins with step 402, in which operational data relating to an algae reactor 116 and/or an algae culture 126 may be received at reactor preparation control processes 15. The operational data may include optimal growth parameters for an algae culture 126 under specified conditions. For example, the optimal spacing of light sources 122 may be 3 inches for certain algae reactors 116. In an alternate embodiments, the spacing of light sources 122 may be 1 inch.

At step 404, one or more reactor surfaces may be prepared. Preparing a reactor surface within a reactor chamber 118 may include sterilizing the surface such as by using steam. Preparing a reactor surface may further include applying an antifouling coating, such as a hydrophobic paint, to prevent water and/or aquatic organisms from attaching to and growing on the prepared surfaces. In some embodiments, a reactor surface may include naturally antifouling characteristics, such as a polycarbonate housing for a light source 122.

At step 406, a reactor control system, which may include a monitoring and control system 146, may be initiated. The monitoring and control system 146 may include and/or communicate with one or more sensors 148, which may be initialized to monitor the characteristics of an algae slurry, growth medium, CO2-containing gas, etc. Sensors 148 may measure one or more of any of temperature, CO2 concentration, algae concentration, light transmission, nutrient concentrations, pH, pressure, etc. In an embodiment, the temperature of the algae slurry may be measured to be 85° F. In another embodiment, the pH may be measured to be 6.5.

At step 408, an algae culture 126 may be prepared. Preparation of an algae culture 126 may include selecting at least one algae strain and may further include one or more treatments to ready the algae culture 126 for introduction into an algae reactor 116 such as exposing the algae culture 126 to ultraviolet light to neutralize any microorganisms such as bacteria, which may inhibit algae growth or be unwanted in the harvested product. In some embodiments, the algae culture 126 may be acquired from a vendor. In other embodiments, the algae culture 126 may be a portion of algae slurry reserved from an algae reactor 116 before, during or after a harvest of the algae biomass. In an embodiment, an algae culture 126 may be reserved from an algae reactor 116 during a harvest, the reserved algae culture is exposed to ultraviolet light, and is then returned to the algae reactor 116.

At step 410, an algae reactor 116 may be filled with at least a growth medium. In an embodiment, the growth medium may be water. Filling the reactor may additionally include introducing an algae culture 126. Filling the reactor may further include introducing a CO2-containing gas, and/or nutrients into the algae reactor 116. In an embodiment, an algae reactor 116 may be filled with water and an algae culture 126.

At step 412, a reactor status may be monitored and returned to the controller 150. The reactor status may include that the reactor is ready to being algae growth operations. Alternatively, the reactor status may include that the reactor is not ready to being algae growth operations. Further, the reactor status may include details about the algae reactor 116, such as which actions have been taken and which actions remain such as the reaction having been sterilized and filled with water, but that the algae culture 126 has not yet been introduced. In an embodiment, the reactor status may be nominal, ready to begin algae growth operations.

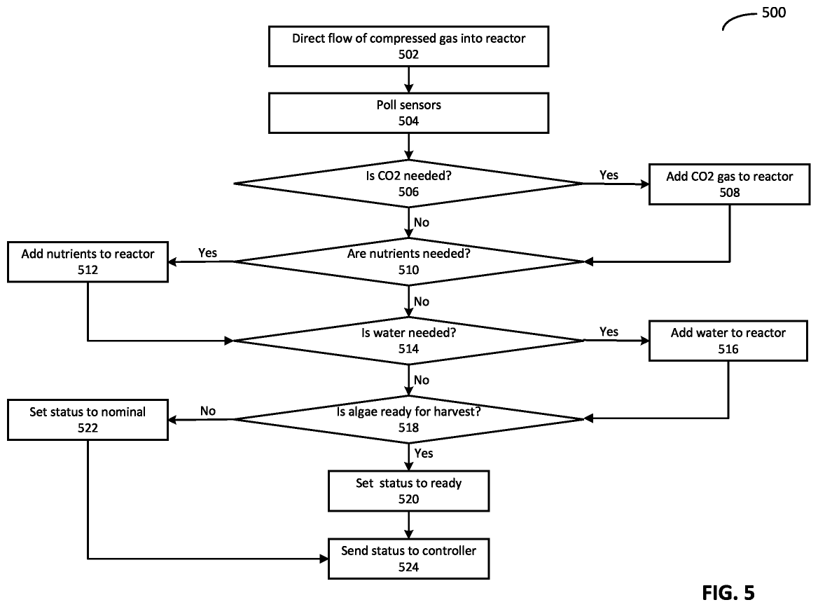

FIG. 5 is a flowchart illustrating an exemplary method for controlling algae growth. The method of FIG. 5 may be performed based on execution of the algae growth control processes 156 by a processor accordance with initiation and call by controller 150.

The process begins at step 502, where a flow of compressed CO2-containing gas may be received in algae reactor 116. The compressed CO2-containing gas may have been cooled, moisture content reduced and optionally been filtered and/or treated to remove one or more contaminant before, during, or after being compressed. The compressed CO2-containing gas may be stored prior to use in an algae reactor 116.

At step 504, one or more sensors 148 may monitor the characteristics of an algae slurry, growth medium, CO2-containing gas, etc., and may be polled for new sensor data. Sensors 148 may measure one or more of any of temperature, CO2 concentration, algae concentration, light transmission, nutrient concentrations, pH, pressure, etc. In an embodiment, the CO2 in solution in the water of the algae slurry may be measured at 6%, nitrogen concentration may be measured at 0.3 mg/l, and the algae reactor 116 may be determined to be at capacity. In an alternate embodiment, the CO2 in solution in the water of the algae slurry may be 1%, nitrogen concentration may be 0.05 mg/l, and the algae reactor 116 may be at 80% capacity.

At step 506, a determination may be made as to whether CO2 is needed. CO2 may be determined to be needed if the CO2 concentration is below a threshold amount. The threshold amount may instead by a range, such that CO2 is not needed if the CO2 concentration is within the target range, but CO2 is needed if the CO2 concentration is below the target range. If CO2 is not needed, determining whether nutrients are needed. The determination of whether CO2 is needed may further include determination of parameters impacting the delivery of CO2, such as determining the optimal bubble size, release rate, etc. which may be impacted by at least the pressure and flow rate of the CO2-containing gas at the bubble generating device 124 which may include a semi-permeable membrane, porous ceramic block, manifold, nozzle, etc. In an embodiment, the light transmittance through an aerated algae slurry with bubbles of a CO2-containing gas of 0.3 mm in size may be measured, and the size of the bubbles may be determined to have increased to 0.5 mm to allow more light to penetrate the algae slurry or reduced to 0.1 mm to improve the CO2 transfer in the fluid medium. In another embodiment, the concentration of CO2 solubilized in solution may be measured directly from one or more sensors 148 in the algae slurry. In another embodiment, it may be determined that the algae slurry is fully saturated with solubilized CO2 when CO2 gas is detected in the exhaust gas from the algae reactor 116. Additional parameters considered may include the agitation of the algae slurry resulting from the rising action of the bubbles of CO2-containing gas.

At step 508, CO2-containing gas may be added to the algae reactor 116 if CO2 is needed. CO2 is added via a bubble generating device 124. In an embodiment, the bubble generating device 124 may be a semipermeable membrane, which when a CO2-containing gas above a pressure threshold may be in contact with the membrane. The pressure threshold being dependent on the membrane and the fluid medium on the opposite side of the membrane. In an embodiment, the membrane may be beneath a column of water, and the pressure threshold is such that the CO2-containing gas can pass through the membrane and into the water column. In a further embodiment, the CO2-containing gas forming bubbles may be less than 0.5 mm in diameter. The surface area of the gas exposed to the medium, such as water, may be maximized, and the rate at which the CO₂ in the CO2-containing gas solubilizes into the medium may also be maximized.

In an embodiment, the CO2 parameters may be adjusted in response to a measurement taken from a light sensor 148 such that the bubbles of CO2-containing gas were increased in size from 0.3 mm to 0.5 mm to increase the light transmitted through the aerated algae slurry. In another embodiment, the volume and/or pressure of CO2-containing gas contacting the bubble generating device 124 may be increased to increase the bubble size being released into the algae reactor 116. In another embodiment, the rate of release of CO2-containing bubbles and/or the size of the bubbles may be decreased in response to CO2 being detected in the exhaust gas from the algae reactor 116 indicating that the algae slurry has exceeded a saturation threshold.

In some embodiments, a CO2 saturated or rich solution of water may be added to the algae reactor 116 instead of or in addition to bubbles of CO2-containing gas. In such embodiments, the CO2-containing gas may be introduced into the water and allowed to solubilize into the water in a vessel or chamber separate from the reactor chamber 118 and may utilize the same methods previously described for introducing a bubble generating gas directly into an algae reactor 116. The algae, in the presence of the CO2 solubilized in the water growth medium and light from one or more light sources 122, undergoes photosynthesis and grows. In some embodiments, the one or more light sources 122 may be arranged within the reactor chamber 118. In another embodiment, the one or more light sources 122 may be arranged outside the reactor chamber 118. In another embodiment, light sources 122 may be arranged both inside and outside the reactor chamber 118.

At step 510, a determination may be made as to whether nutrients are needed. Nutrients may include any of nitrogen, phosphorus, and potassium and any number of additional trace elements. Nutrients may be required if one or more sensors measuring one or more nutrient concentrations in the medium are below a threshold value. Alternatively, the threshold may instead be a range, and nutrients are needed if the measured value of one or more nutrients are outside the target range.

At step 512, one or more nutrients may be added to the algae reactor 116 if the one or more nutrients are needed. In an embodiment, the nutrient concentration for nitrogen may be determined to be low, and ammonium nitrate may be added to increase the amount of nitrogen solubilized in the growth medium and available to the algae. In another embodiment, the amount of potassium may be low, and potassium sulphate may be added to increase the concentration of potassium available to the algae in the reactor chamber 118.

At step 514, a determination may be made as to whether water or another growth medium is needed to be added to the reactor chamber 118. Water is needed if the water level is below a threshold value. The threshold value may be a height within the reactor chamber 118, a percentage of the volume of the reactor chamber 118, and/or a ratio of water to algae.

At step 516, water or another growth medium may be added to the reactor chamber 118 if more water is needed. Adding water may include introducing water from a water holding tank 114 and/or a municipal water source. In an embodiment, the water may be added from a water holding tank 114. The water originates from another process, such as from drying a CO2-containing gas, or drying algae biomass by a harvesting system 132, such as by a biomass membrane separator 134, or biomass steam dryer 136.

At step 518, a determination may be made as to whether the algae is ready for harvest. The algae may be ready for harvest by a harvesting system 132 if the density of the algae in the medium, such as water, is above a threshold concentration. The concentration may be determined by one or more of the penetration of light through the algae slurry, viscosity of the algae slurry, ratio of algae to water, mass of the algae slurry, volume of the algae slurry, etc. In some embodiments, the algae is determined to be ready for harvest when the growth rate of the algae begins to slow, or slows to a threshold rate.

At step 520, the reactor status may be set to “ready for harvest” if it is determined that the algae is ready for harvest. In an embodiment, the algae may be ready for harvest, as the growth rate of the algae has begun to slow, and the penetration of light through the algae slurry is below 20%, where 100% is the amount of light penetration through water with no algae present.

At step 522, the reactor status may be set to “nominal,” thereby indicating that the reactor is operating within normal parameters including at least CO2 and/or nutrients dissolved in the medium, and that the algae is not ready for harvest. If the reactor status is nominal, the algae is allowed to continue to grow, while exposed to light from one or more light sources 122.

At step 524, the reactor status including an algae growth status may be returned to the controller 150. In an embodiment, a nominal reactor status may be returned. In an alternate embodiment, a reactor status indicating that the algae is ready to be harvested may be returned.

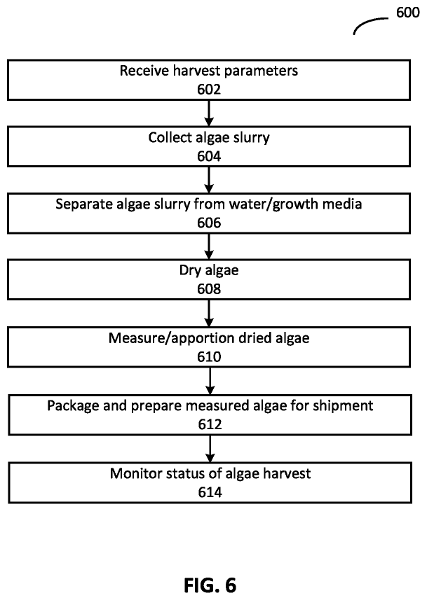

FIG. 6 is a flowchart illustrating an exemplary method for harvesting and processing algae products. The method of FIG. 6 may be performed based on execution of the harvest control processes 158 by a processor accordance with initiation and call by controller 150.

The process begins with at step 602, in which harvest data may be received from the controller 150. The harvest data may include one or more of a processing procedure and/or parameters, such as a target moisture content. In an embodiment, the harvest data may include a target moisture content of less than 5%.

At step 604, an algae slurry may be collected from one or more algae reactors 116. The algae slurry may include algae and a growth medium. In an embodiment, the growth medium may be water. The algae slurry may be pumped, poured, drained, scooped, or otherwise removed from an algae reactor 116 and transported to a harvesting system 132. In some embodiments, some of the algae slurry may be retained and used as an algae culture 126. Such retained algae may be treated such as by using UV light and other methods to ensure that undesirable microbes and other potential contaminants are not retained.

At step 606, water or growth media may be separated from an algae slurry. In an embodiment, the separation of the growth medium, such as water, may be performed using a biomass membrane separator 134 from the algae slurry. In some implementations, more than 50% of the water in the algae slurry may be removed. The separated water may be retained in a water holding tank 114 and may be recycled through the algae growth cycle. In some embodiments, the separated water may be filtered and/or otherwise treated prior to storage and/or reuse or discharge.

At step 608, the algae may be dried to a desired moisture content, such as may have been indicated in harvest data received from the controller 150. In an embodiment, a biomass steam dryer may be used to remove the remaining water from the algae. In some implementations, remaining water may be removed from the biomass until no more than 5% remains. In alternate embodiments, a higher or lesser target moisture content may be used as required by the intended use. The intended use may be determined by a purchaser of the dried algae.

At step 610, the dried algae may be measured and portioned, and in step 612, the dried algae may be packaged and prepared for shipment. In an embodiment, a biomass packager may be a bagger, and the dried algae is weighted and portioned into one or more bags. In an alternate embodiment, the dried algae may be portioned and packaged in other containers such as canisters, or bulk movable storage such as a truck trailer. The container may depend upon a customer's requirements and/or the parameters and/or characteristics of the dried algae. For example, algae with a higher moisture content than 5% may be ill suited to storage in bags, and therefore may be loaded into bottles, totes, tanks, etc. The measured/portioned and packaged algae may further be prepared for shipment, which may include loading bags and/or containers of dried algae on one or more pallets and further storing or transporting the algae via trucks, trains, or other means of transportation.

At step 614, a harvest status may be monitored and returned to the controller 150. In an embodiment, the harvest status may indicate the quantity of algae which has been harvested. The harvest status may further include data from analysis of the harvested algae such as the content of moisture, protein, carbohydrates, lipids, etc.

FIG. 7 illustrates an exemplary computing system 700 that may be used to implement an embodiment of the present invention. The computing system 700 of FIG. 7 may include one or more processors 710 and memory 720. Main memory 720 stores, in part, instructions and data for execution by processor 710. Main memory 720 can store the executable code when in operation. The system 700 of FIG. 7 may further include a mass storage device 730, portable storage device 740 (e.g., media drive), output devices 750, user input devices 760, a graphics display system 770, and peripheral devices 780.

The components shown in FIG. 7 are depicted as being connected via a single bus 790. However, the components may be connected through one or more data transport means. For example, processor unit 710 and main memory 720 may be connected via a local microprocessor bus, and the mass storage device 730, peripheral device(s) 780, portable storage device 740, and graphics display system 770 may be connected via one or more input/output (I/O) buses.

Mass storage device 730, which may be implemented with a magnetic disk drive or an optical disk drive, is a non-volatile storage device for storing data and instructions for use by processor unit 710. Mass storage device 730 can store the system software for implementing embodiments of the present invention for purposes of loading that software into main memory 720.

Portable storage device 740 operates in conjunction with a portable non-volatile storage medium, such as a floppy disk, compact disk or Digital video disc, to input and output data and code to and from the computer system 700 of FIG. 7. The system software for implementing embodiments of the present invention may be stored on such a portable medium and input to the computer system 700 via the portable storage device 740.

Input devices 760 provide a portion of a user interface. Input devices 760 may include an alpha-numeric keypad, such as a keyboard, for inputting alpha-numeric and other information, or a pointing device, such as a mouse, a trackball, stylus, or cursor direction keys. Additionally, the system 700 as shown in FIG. 7 includes output devices 750. Examples of suitable output devices include speakers, printers, network interfaces, and monitors.

Display system 770 may include a liquid crystal display (LCD) or other suitable display device. Display system 770 receives textual and graphical information, and processes the information for output to the display device.

Peripheral devices 780 may include any type of computer support device to add additional functionality to the computer system. For example, peripheral device(s) 780 may include a modem or a router.

The components contained in the computer system 700 of FIG. 7 are those typically found in computer systems that may be suitable for use with embodiments of the present invention and are intended to represent a broad category of such computer components that are well known in the art. Thus, the computer system 700 of FIG. 7 can be a personal computer, hand held computing device, telephone, mobile computing device, workstation, server, minicomputer, mainframe computer, or any other computing device. The computer can also include different bus configurations, networked platforms, multi-processor platforms, etc. Various operating systems can be used including Unix, Linux, Windows, Macintosh OS, Palm OS, and other suitable operating systems.

The functions performed in the processes and methods may be implemented in differing orders. Furthermore, the outlined steps and operations are only provided as examples, and some of the steps and operations may be optional, combined into fewer steps and operations, or expanded into additional steps and operations without detracting from the essence of the disclosed embodiments.

What is claimed is:

1. A system for biomass-based capture of carbon dioxide, the system comprising:
a connection to a gas source;
a reactor chamber that receives gas from the gas source via the connection, wherein an algae suspension within the reactor chamber is exposed to the received gas;
one or more sensors that measures one or more characteristics of the algae suspension in real-time; and
a control system that:
receives the measured characteristics from the sensors in real-time,
compares the measured characteristics to one or more predetermined target characteristics, and
generates an initiation signal based on the comparison, wherein the initiation signal is associated with a recipient device.

2. The system of claim 1, further comprising a bubbling apparatus configured to generate bubbles that disperse the gas from the gas source within the algae suspension within the reactor chamber, wherein the bubbling apparatus controls a rate at which the bubbles are generated.

3. The system of claim 2, wherein the bubbling apparatus is the recipient device that receives the initiation signal and changes the rate at which the bubbles are generated based on the initiation signal.

4. The system of claim 1, further comprising one or more light sources that generate light within the reactor chamber.

5. The system of claim 4, wherein the light sources include one or more light-emitting diodes (LEDs), and further comprising an LED wavelength control system that controls a wavelength or intensity of light generated by the LEDs.

6. The system of claim 5, wherein the LED wavelength control system is the recipient device that receives the initiation signal and changes the wavelength or intensity of light generated by the LEDs based on the initiation signal.

7. The system of claim 1, further comprising a compressor that controls a pressure or flow rate of the gas introduced into the reactor chamber.

8. The system of claim 7, wherein the compressor is the recipient device that receives the initiation signal and changes the pressure or flow rate of the gas introduced into the reactor chamber based on the initiation signal.

9. The system of claim 1, further comprising a nutrient reservoir that injects one or more nutrient solutions into the algae suspension.

10. The system of claim 9, wherein the nutrient reservoir is the recipient device that receives the initiation signal and changes delivery of one or more of the nutrient solutions injected into the algae suspension based on the initiation signal.

11. The system of claim 1, wherein the algae suspension includes algae biomass within a liquid media, and further comprising a harvesting system configured to separate the algae biomass from the liquid media.

12. The system of claim 11, wherein the predetermined target characteristics include a target density of the algae suspension, and wherein harvesting system is the recipient device that receives the initiation signal and initiates separation of the algae biomass from the liquid media based on the initiation signal.

13. A method for biomass-based capture of carbon dioxide, the method comprising:
receiving gas from a gas source via a connection to the gas source, the gas received within a reactor chamber;
exposing an algae suspension within the reactor chamber to the received gas;
measuring one or more characteristics of the algae suspension in real-time via one or more sensors;
comparing the measured characteristics to one or more predetermined target characteristics; and
generating an initiation signal based on the comparison, wherein the initiation signal is associated with a recipient device.

14. The method of claim 13, further comprising generating bubbles via a bubbling apparatus, wherein the bubbles disperse the gas from the gas source within the algae suspension within the reactor chamber, wherein a rate at which the bubbles are generated is controlled by the bubbling apparatus.

15. The method of claim 14, further comprising sending the initiation signal to the bubbling apparatus, wherein the bubbling apparatus changes the rate at which the bubbles are generated based on the initiation signal.

16. The method of claim 13, further comprising generating light within the reactor chamber via one or more light sources.

17. The method of claim 16, wherein the light sources include one or more light-emitting diodes (LEDs), and further comprising controlling a wavelength or intensity of light generated by the LEDs via an LED wavelength control system.

18. The method of claim 17, further comprising sending the initiation signal to the LED wavelength control system, wherein the LED wavelength control system receives the initiation signal and changes the wavelength or intensity of light generated by the LEDs based on the initiation signal.

19. The method of claim 13, further comprising controlling a pressure or flow rate of the gas introduced into the reactor chamber via a compressor.

20. The method of claim 19, further comprising sending the initiation signal to the compressor, wherein the compressor receives the initiation signal and changes the pressure or flow rate of the gas introduced into the reactor chamber.

21. The method of claim 13, further comprising injecting one or more nutrient solutions from a nutrient reservoir into the algae suspension.

22. The method of claim 21, further comprising sending the initiation signal to the nutrient reservoir, wherein the nutrient reservoir receives the initiation signal and changes delivery of one or more of the nutrient solutions injected into the algae suspension.

23. The method of claim 13, wherein the algae suspension includes algae biomass within a liquid media, and further comprising separating the algae biomass from the liquid media using a harvesting system.

24. The method of claim 23, wherein the predetermined target characteristics include a target density of the algae suspension, and further comprising sending the initiation signal to the harvesting system, wherein the harvesting system receives the initiation signal and initiates separation of the algae biomass from the liquid media.

25. A non-transitory, computer-readable storage medium, having embodied thereon a program executable by a processor to perform a method for biomass-based capture of carbon dioxide, the method comprising:
receiving gas from a gas source via a connection to the gas source, the gas received within a reactor chamber;
exposing an algae suspension within the reactor chamber to the received gas;
measuring one or more characteristics of the algae suspension in real-time via one or more sensors;
comparing the measured characteristics to one or more predetermined target characteristics; and
generating an initiation signal based on the comparison, wherein the initiation signal is associated with a recipient device.
